(19) United States
(12) Patent Application Publication
(10) Pub. No.: US 2018/0030485 A1
(43) Pub. Date: Feb. 1, 2018

(54) PRODUCTION METHOD FOR OIL AND FAT

(71) Applicant: FUJI OIL HOLDINGS INC., Osaka (JP)

(72) Inventor: Shimpei WATANABE, Kaizuka-shi, Osaka (JP)

(73) Assignee: FUJI OIL HOLDINGS INC., Osaka (JP)

(21) Appl. No.: 15/552,948

(22) PCT Filed: Feb. 23, 2016
(86) PCT No.: PCT/JP2016/055279

§ 371 Date: Aug. 23, 2017

(30) Foreign Application Priority Data
Feb. 26, 2015 (JP) 2015-036168
Feb. 19, 2016 (JP) 2016-029432

(51) Int. Cl. C12P 7/64; C11C 1/10; C11C 3/10

(52) U.S. Cl. CPC C12P 7/64; C11C 3/10; C11C 1/10

(57) ABSTRACT

The present invention provides a commercially-viable-level, highly efficient production method for oil and fat that are rich in USU.

### TECHNICAL FIELD

The present invention relates to a process for producing a fat rich in USU including using a 1,3-position interesterification reaction, where S is a saturated fatty acid with C16 to C24, U is an unsaturated fatty acid with C18, and USU is a triglyceride whose fatty acids at 1,3-positions are U and fatty acid at 2-position is S.

### BACKGROUND ART

Patent Document 1 discloses a cocoa butter equivalent containing 40 to 100% by weight of mixed acid group triglyceride in which a saturated fatty acid having carbon atoms 16 to 22 is bound to a glycerin at 2-position and an unsaturated fatty acid having carbon atoms 16 to 18 and one unsaturated bond is bound to the glycerin at 1,3-positions as constituent fatty acids.

Patent Document 1 discloses that this mixed acid group triglyceride exhibits the following peculiar physical properties. The mixed acid group triglyceride forms a peculiar crystalline structure with a cocoa butter. By combining the mixed acid group triglyceride as a raw material of a chocolate, a bloom is not observed at all without performing a tempering. The obtained chocolate shows that resistivity of a crystal against a pressure is significantly small although it shows a melting point almost same as that of ordinary chocolates.

As an example of a method for producing OStO (where St is stearic acid and O is oleic acid), which is one kind of the mixed acid group triglyceride, Example 1 of Patent Document 2 discloses a method including the steps of interesterification reaction of fully hydrogenated soybean oil and ethyl oleate using 1,3-position-selective enzyme, molecular distillation, fractionation, and refining.

### PRIOR ART DOCUMENTS

### Patent Documents

Patent Document 1: JP H04-135453 A

Patent Document 2: JP 2002-65162 A

### SUMMARY OF INVENTION

### Problems to be Solved by Invention

The inventor has found the following problems. In the method of producing a fat rich in a triglyceride of interest by an interesterification reaction of fat and fatty acid or lower alcohol ester thereof (hereinafter, referred to as “fatty acid ester etc.”), high concentration of U, which is relatively expensive material, must be used in large amount in order to produce a fat containing USU by a method of using 1,3-position interesterification reaction of a fat containing S as major component and a fatty acid ester containing U as major component. However, lower concentration of U generated after the interesterification reaction cannot be concentrated to the level as the starting material with simple method. That is, the U is non-reusable in circle, and thus, used for a low-value-added product or discarded. It results extremely high cost of the production. That is, an object of the present invention is to provide a process to easily and efficiently produce USU-containing fat to provide the USU-containing fat at a commercially available level.

### Means for Solving the Problems

The inventor has found the followings. The use of fat containing a saturated fatty acid with C20 to 24 as raw material fat for an interesterification reaction ensures easily separating the saturated fatty acid with C20 to 24, which is liberated derived from the raw material fat after the interesterification reaction, from an unreacted U by a distillation using a difference between respective boiling points. Additionally, the U concentrated by the separation may be effectively circulated for reuse as a raw material fatty acid for the interesterification reaction. From these findings, the inventor has completed an efficient and easy process for producing a USU-containing fat at a commercially available level. The inventor has also found that the use of a fully hydrogenated oil of high erucic rapeseed oil as the raw material fat and an ethyl oleate as the raw material fatty acid may obtain a pure OStO containing only a small amount of OBO (B is behenic acid and O is oleic acid). Thus, the inventor has completed the efficient process for producing a high-purity OStO fat at a commercially available level.

The inventor has also found the followings. The use of fat containing palmitic acid as the raw material fat for the interesterification reaction ensures easily separating palmitic acid, which is liberated derived from the raw material fat after the interesterification reaction, from the unreacted U by the distillation using a difference between respective boiling points. Additionally, the U concentrated by the separation may be effectively circulated for reuse as the raw material fatty acid for the interesterification reaction. From these findings, the inventor has completed an efficient and easy process for producing a USU-containing fat at a commercially available level. The inventor has also found that the use of a fully hydrogenated oil of palm mid fraction as the raw material fat and the ethyl oleate as the raw material fatty acid may obtain the pure OStO containing only a small amount of OPO (P is palmitic acid and O is oleic acid). Thus, the inventor has completed the efficient process for producing the high-purity OStO fat at a commercially available level.

That is, the first aspect of the present invention is a process for producing a fat composition containing USU, including:

(1) a step of mixing (a) raw material fat containing 80% by weight or more of (S) saturated fatty acid with C16 to C24, where the (a) raw material fat contains 10 to 70% by weight of saturated fatty acid with C20 to C24 or 10 to 70% by weight of palmitic acid, and (b) raw material fatty acid or lower alcohol ester thereof containing (U) unsaturated fatty acid with C18 as major component;

(2) a step of subjecting the mixture obtained in step (1) to an interesterification reaction using a 1,3-position-specific lipase;

(3) a step of separating the fat composition containing 30% by weight or more of USU as a triglyceride fraction, and a fraction of fatty acid or lower alcohol ester thereof; and

(4) a step of separating the unreacted (b) raw material fatty acid or lower alcohol ester thereof, to circulate a part or all of the recovered unreacted (b) raw material fatty acid or lower alcohol ester thereof as the (b) in step (1) for reuse,

where USU is a triglyceride whose fatty acids at 1,3-positions are U and fatty acid at 2-position is S.

The second aspect of the present invention is the process for producing the fat composition according to the first aspect, where at least one of the separations at step (3) and step (4) is performed by a distillation.

The third aspect of the present invention is the process for producing the fat composition according to the first aspect or the second aspect, where the (a) raw material fat contains 80% by weight or more of S and 10 to 70% by weight of the saturated fatty acid with C20 to 24 in the constituent fatty acid.

The fourth aspect of the present invention is the process for producing the fat composition according to the third aspect, where a part or all of the (a) raw material fat in step (1) is a fully hydrogenated oil of high erucic rapeseed oil, and where the (b) raw material fatty acid or lower alcohol ester thereof contains 70% by weight or more of oleic acid.

The fifth aspect of the present invention is the process for producing the fat composition according to the first aspect or the second aspect, where the (a) raw material fat contains 80% by weight or more of S and 10 to 70% by weight of palmitic acid in the constituent fatty acid.

The sixth aspect of the present invention is the process for producing the fat composition according to the fifth aspect, where a part or all of the (a) raw material fat in step (1) is a fully hydrogenated oil of palm mid fraction, and where the (b) raw material fatty acid or lower alcohol ester thereof contains 70% by weight or more of oleic acid.

The seventh aspect of the present invention is the process for producing the fat composition according to any one of the first to the six aspects, where the triglyceride fraction separated in step (3) is fractionated, and a part or all of the obtained high-melting point fraction is circulated for reuse as the (a) in step (1).

The eighth aspect of the present invention is the process for producing the fat composition according to any one of the first to seventh aspects, where the triglyceride fraction separated in step (3) is fractionated to obtain the fat composition containing 40% by weight or more of USU as a low-melting point fraction or a mid-melting point fraction.

### Effects of the Invention

The present invention enables to produce fat rich in USU, especially OStO, at relatively low cost. This ensures a wide variety of applications of fat rich in OStO to food products whose application development has not been advanced up to the present due to the expensiveness. Additionally, since a fatty acid ester etc. may be circulated for reuse, this process for producing fat is environmentally friendly.

### MODE FOR CARRYING OUT THE INVENTION

Hereinafter, the present invention will be described in detail.

The (a) raw material fat of the present invention contains 80% by weight or more of S, and 10 to 70% by weight of saturated fatty acid with C20 to 24, or 10 to 70% by weight of palmitic acid, in a constituent fatty acid.

The (a) raw material fat according to the first aspect of the present invention may contain 80% by weight or more of (S) saturated fatty acid with C16 to C24, and 10 to 70% by weight of saturated fatty acid with C20 to 24, in the constituent fatty acid. The (a) preferably contains 90% by weight or more, more preferably 95% by weight or more, and further preferably 98% by weight or more of S. The (a) preferably contains 20% by weight or more, further preferably 30% by weight or more, and the most preferably 40% by weight or more of saturated fatty acid with C20 to 24. And, the (a) preferably contains 60% by weight or less, further preferably 55% by weight or less of saturated fatty acid with C20 to 24. When the S in the constituent fatty acid of the (a) raw material fat according to the first aspect of the present invention is less than 80% by weight, this triglyceride content in the fat composition containing USU may be reduced.

When the saturated fatty acid with C20 to 24 in the constituent fatty acid of the (a) raw material fat according to the first aspect of the present invention is less than 10% by weight, the amount of the saturated fatty acid with C20 to 24 discharged outside the system is small, and thereby possibly failing to obtain the sufficient level of USU fat production efficiency. In the case of producing OStO fat, when the saturated fatty acid with C20 to 24 is more than 70% by weight, the St content is relatively reduced, and thereby possibly failing to obtain the sufficient level of OStO fat production efficiency.

In the case of where the objective USU fat is high-purity OStO fat in the first aspect of the present invention, the (a) raw material fat preferably contains the saturated fatty acids with C20 to 24 much at 1, 3-positions and little at 2-position. In this case, preferably 80% by weight or more, more preferably 90% by weight or more, further preferably 95% by weight or more, most preferably 98 weight % or more of the saturated fatty acids with C20 to 24 in the constituent fatty acid of the (a) raw material fat are present at the 1, 3-positions. The use of such raw material fat ensures easily separating the saturated fatty acid with C20 to 24, which is liberated derived from the raw material fat after the interesterification reaction, from the unreacted U, by using the difference between the respective boiling points by the distillation. The U concentrated through the separation may be effectively circulated for reuse as the raw material fatty acid for the interesterification reaction.

A content ratio of the saturated fatty acid with C20 to 24 to the S content is preferably 0.2 or more, more preferably 0.3 or more, further preferably 0.4 or more, and preferably 0.85 or less, more preferably 0.75 or less, and further preferably 0.70 or less, in the constituent fatty acid of the (a) raw material fat according to the first aspect of the present invention. When this content ratio is less than the lower limit, an amount of the discharged saturated fatty acid with C20 to 24 is small, and thereby possibly failing to obtain the sufficient level of USU fat production efficiency. In the case of producing OStO fat, when this content ratio exceeds the upper limit, the St content is relatively reduced, and thereby possibly failing to obtain the sufficient level of OStO fat production efficiency.

The (a) raw material fat of the first aspect of the present invention is not particularly limited as long as the above-described requirements for the fatty acid composition are met. Examples of the (a) raw material fat include fully hydrogenated oil such as fully hydrogenated high-erucic rapeseed oil, fully hydrogenated fish oil, and fully hydrogenated jojoba oil, and interesterified oil or fractionated oil in which one or more of the above described oils are used as a raw material. When USU fat is intended to high-purity OStO fat, the (a) raw material fat is most preferably the fully hydrogenated high-erucic rapeseed oil.

The (a) raw material fat according to the second aspect of the present invention may contain 80% by weight or more of S and 10 to 70% by weight of palmitic acid. Further, the (a) raw material fat contains preferably 90% by weight or more, more preferably 95% by weight or more, and further preferably 98% by weight or more of S. And, the (a) raw material fat contains preferably 20% by weight or more, further preferably 30% by weight or more, and most preferably 40% by weight or more of palmitic acid. The palmitic acid content is preferably 60% by weight or less, and further preferably 55% by weight or less. When the S in the constituent fatty acid of the (a) raw material fat according to the second aspect of the present invention is less than 80% by weight, this triglyceride content in the fat composition containing USU may be reduced. When the palmitic acid of the (a) raw material fat according to the second aspect of the present invention is less than 10% by weight, the amount of the palmitic acid discharged outside the system is small, and thereby possibly failing to obtain the sufficient level of USU fat production efficiency. In the case of producing OStO fat, when the palmitic acid is more than 70% by weight, the St content is relatively reduced, and thereby possibly failing to obtain the sufficient level of OStO fat production efficiency. In the case of where the objective USU fat is high-purity OStO fat in the second aspect of the present invention, the (a) raw material fat preferably contains palmitic acid much at 1, 3-positions and little at 2-position. In this case, preferably 80% by weight or more, more preferably 90% by weight or more, further preferably 95% by weight or more, most preferably 98 weight % or more of the palmitic acid in the constituent fatty acid of the (a) raw material fat are present at the 1, 3-positions. The use of such raw material fat ensures easily separating the palmitic acid, which is liberated derived from the raw material fat after the interesterification reaction, from the unreacted U, by using the difference between the respective boiling points by the distillation. The U concentrated through the separation may be effectively circulated for reuse as the raw material fatty acid for the interesterification reaction.

A content ratio of the palmitic acid to the S content is preferably 0.2 or more, more preferably 0.3 or more, further preferably 0.4 or more, and preferably 0.85 or less, more preferably 0.75 or less, and further preferably 0.70 or less, in the constituent fatty acid of the (a) raw material fat according to the second aspect of the present invention. When this content ratio is less than the lower limit, an amount of the discharged palmitic acid is small, and thereby possibly failing to obtain the sufficient level of USU fat production efficiency. In the case of producing OStO fat, when this content ratio exceeds the upper limit, the St content is relatively reduced, and thereby possibly failing to obtain the sufficient level of OStO fat production efficiency.

The (a) raw material fat of the second aspect of the present invention is not particularly limited as long as the above-described requirements for the fatty acid composition are met. Examples of the (a) raw material fat include fully hydrogenated oil of palm mid fraction, and fully hydrogenated Chinese tallow oil, and interesterified oil or fractionated oil in which one or more of the above described oils are used as a raw material. And, the (a) raw material fat is most preferably the fully hydrogenated oil of palm mid fraction.

The (b) raw material fatty acid of the present invention is not particularly limited as long as it is a raw material fatty acid ester etc. containing unsaturated fatty acid with 18 carbon atoms as the main component. From the viewpoint of abundance in nature and easy availability, the (b) is preferably oleic acid or lower alcohol ester thereof, more preferably ethyl oleate. The content of the oleic acid is preferably 70% by weight or more, more preferably 75% by weight or more.

A raw material other than the (a) raw material fat and the (b) raw material fatty acid ester etc. may be added to the raw material mixture in step (1) of the present invention unless the raw material mixture does not degrade the effects of the present invention. A total amount of the (a) raw material fat and the (b) raw material fatty acid ester etc. in the raw material mixture is preferably 80% by weight or more, more preferably 90% by weight or more, further preferably 95% by weight or more, and most preferably 98% by weight or more.

A lipase produced by microorganisms such as genus Rhizopus, genus Aspergillus, and genus Mucor may be used in the selective interesterification using 1,3-position-specific lipase in step (2) of the present invention. Any lipases other than the above-described lipases may be used as long as the lipases have a similar property to these lipases. Such lipase is commercially available, and for example, Amano A (Amano Enzyme Inc.), and Lipozyme (Novo Nordisk Pharma Ltd.) are used. The type of usage of the lipase is not particularly limited, and the lipase is preferably used with immobilized to a carrier by a known method from a viewpoint of efficiency. Additionally, a chemically modified enzyme is preferable used under an organic solvent. This reaction may be performed by a batch method using a stir tank and a continuation method using a filled reactor.

The raw material mixture subjected to the enzyme reaction of the selective interesterification reaction using 1,3-position-specific lipase in step (2) of the present invention is preferably bleached and deodorized by a known method before the enzyme reaction in order to reduce the enzyme inactivation as much as possible. A water content in the raw material mixture is desired to be adjusted low in order to reduce a hydrolysis reaction as much as possible to reduce a generation of diglyceride, but it is desired to be adjusted high in order to enhance a reaction rate. The water content is desired to be adjusted to 10 to 300 ppm, preferably 20 to 200 ppm, and further preferably 30 to 100 ppm. A period of the enzyme reaction is not particularly limited as long as a sufficient interesterification reaction rate is achieved, and is preferably two hours to four days. A temperature of the enzyme reaction is desirable to be 30 td 80° C., more preferably 35 to 65° C., and further preferably 40 to 55° C. from the viewpoints of maintaining an enzyme activation long while securing the sufficient enzyme reaction rate and reducing the generation of isomer triglyceride as much as possible. When the continuation method using the filled reactor is applied to step (2) of the present invention, the reaction temperature is preferably a temperature at which a crystal precipitation does not occur during the reaction to avoid an obstruction inside the reactor. However, the crystal precipitation temperature varies depending on a mixing ratio of the (a) raw material fat mainly containing saturated fatty acid, which has a comparatively high-melting point, to the (b) raw material fatty acid ester etc. mainly containing unsaturated fatty acid, which has a low-melting point. Thus, the mixing ratio in step (1) is advantageous when the (b) raw material fatty acid ester etc. is larger, and thus, the ratio of the (b) raw material fatty acid ester etc. in the raw material mixture is preferably 35% by weight or more, more preferably 50% by weight or more, and further preferably 60% by weight or more. In addition, the small amount of the (a) raw material fat reduces a production amount of fat composition as a triglyceride fraction obtained in step (3), resulting in the deterioration of production efficiency. From this point, the ratio of the (a) raw material fat in the raw material mixture is preferably 5% by weight or more, more preferably 10% by weight or more, and further preferably 20% by weight or more.

A fractionation or a distillation is applicable to the separation into the triglyceride fraction and the fatty acid ester etc. fraction in step (3) of the present invention, and the distillation is preferable. The condition for distillation is not particularly limited as long as the condition enables to separate the triglyceride fraction and the fatty acid ester etc. fraction. The distillation temperature is preferably 180° C. or more, more preferably 200° C. or more, further preferably 210° C. or more, and the most preferably 220° C. or more. In addition, the distillation temperature is preferably 280° C. or less, more preferably 270° C. or less, further preferably 260° C. or less, and the most preferably 250° C. or less. The degree of vacuum is preferably 0.2 torr or more, more preferably 0.5 torr or more, and further preferably 1 torr or more. In addition, the degree of vacuum is 10 torr or less, more preferably 7 torr or less, further preferably 5 torr or less, and the most preferably 3 torr or less.

The triglyceride fraction obtained in step (3) of the present invention is a fat composition containing 30% by weight or more, preferably 40% by weight or more, further preferably 50% by weight or more, and the most preferably 60% by weight or more of USU. Increasing the mixing ratio of the (b) raw material fatty acid in step (1) and increasing the interesterification reaction rate in step (2) ensure the increase in this USU content.

The fatty acid ester etc. fraction, which is obtained in step (3) of the present invention, is a mixture of the unreacted raw material fatty acid ester etc. and the fatty acid ester etc. derived from the 1, 3-positions of the (a) raw material fat. Then, the unreacted (b) raw material fatty acid ester etc. is separated in the subsequent step (4). A distillation, which is the separation using the difference between the boiling points, and a combination of the distillation and another separation method such as a preliminary execution of a rough separation using the difference between the melting point by the fractionation prior to the distillation may be used as the method for separation. The distillation temperature is preferably 180° C. or more, more preferably 200° C. or more, further preferably 210° C. or more, and the most preferably 220° C. or more. In addition, the distillation temperature is preferably 280° C. or less, more preferably 270° C. or less, further preferably 260° C. or less, and the most preferably 250° C. or less. The degree of vacuum is preferably 0.2 torr or more, more preferably 0.5 torr or more, and further preferably 1 torr or more. In addition, the degree of vacuum is preferably 10 torr or less, more preferably 7 torr or less, further preferably 5 torr or less, and the most preferably 3 torr or less.

In the first aspect, the unreacted raw material fatty acid ester etc. such as the unsaturated fatty acid ester etc. with C18 and the fatty acid with less than C20 (mainly containing the saturated fatty acid) or the lower alcohol ester among the fatty acid derived from the (a) raw material fat are separated into low-boiling point fractions and are recovered by the distillation in step (4). When the content of unsaturated fatty acid ester etc. with C18 as this low-boiling point fraction is not significantly low compared with the (b) raw material fatty acid ester etc., a part or all of the low-boiling point fraction is circulated for reuse as the raw material mixture in step (1). When the content is significantly low, prior to this circulation for reuse, the mixed fatty acid with less than C20 (mainly containing the saturated fatty acid) or the lower alcohol ester is removed by a known method such as fractionation and adsorption. Thus, after the purity of the unsaturated fatty acid ester etc. with C18 is increased, the low-boiling point fraction may be circulated for reuse for the raw material mixture in step (1). Meanwhile, the fatty acid ester etc. with C20 to 24 among the fatty acid derived from the (a) raw material fat is recovered to high-boiling point fraction.

In the second aspect, the unreacted raw material fatty acid ester etc. such as the unsaturated fatty acid ester etc. with C18 and the fatty acid with more than C16 (mainly containing the saturated fatty acid) or the lower alcohol ester among the fatty acid derived from the (a) raw material fat are separated into high-boiling point fractions and are recovered by the distillation in step (4). When the content of unsaturated fatty acid ester etc. with C18 as this low-boiling point fraction is not significantly low compared with the (b) raw material fatty acid ester etc., a part or all of the low-boiling point fraction is circulated for reuse as the raw material mixture in step (1). When the content is significantly low, prior to this circulation for reuse, the mixed fatty acid with more than C16 (mainly containing the saturated fatty acid) or the lower alcohol ester is removed by a known method such as fractionation and adsorption. Thus, after the purity of the unsaturated fatty acid ester etc. with C18 is increased, the low-boiling point fraction may be circulated for reuse for the raw material mixture in step (1). Meanwhile, the fatty acid ester etc. of palmitic acid among the fatty acid derived from the (a) raw material fat is recovered to low-boiling point fraction.

The order of the distillations in step (3) and step (4) of the present invention is not particularly limited. For example, it is preferable that the distillation in step (3), in which the triglyceride fraction and the fraction of the fatty acid ester etc. having the large difference in the boiling points are distilled and separated, is performed and then the latter fraction is further subjected to the distillation in step (4), in which a fraction of the by-product fatty acid ester etc. derived from the 1, 3-positions of the raw material fat and a fraction of the unreacted fatty acid ester etc. having the comparatively small difference in the boiling points are distilled and separated. However, depending on a convenience of the steps of these distillations, step (3) may be performed after step (4), and steps (3) and (4) may be performed simultaneously. Examples of the distillation in steps (3) and (4) include single distillation, steam distillation, thin film distillation, molecular distillation, and rectification. The distillation in step (4) is preferably rectification.

The triglyceride fraction, which is separated in step (3) of the present invention, is fractionated by a known method such as solvent fractionation to produce a low-melting point fraction or a mid-melting point fraction in order to increase the USU content to 40% by weight or more, preferably 60% by weight or more, and further preferably 80% by weight or more.

A part or all of a fractionated high-melting point fraction, which is secondarily produced at the fractionation, may be circulated for reuse for the raw material mixture in step (1). In this case, the process for producing USU fat further enhancing the production efficiency.

Like the first aspect of the present invention, the use of the fully hydrogenated high-erucic rapeseed oil as the (a) raw material fat and the use of the fatty acid ester etc. containing 70% by weight or more of oleic acid as the (b) raw material fatty acid ensures obtaining the high purity OStO fat that contains only a small amount of OBO. Accordingly, this process is especially advantageous from the viewpoint of the process for producing the high purity OStO fat with a commercially available level of production efficiency. Like the second aspect of the present invention, the use of the fully hydrogenated palm mid fraction as the (a) raw material fat and the use of the fatty acid ester etc. containing 70% by weight or more of oleic acid as the (b) raw material fatty acid ensures obtaining the high purity OStO fat that contains only a small amount of OPO. Accordingly, this process is especially advantageous from the viewpoint of the process for producing the high purity OStO fat with a commercially available level of production efficiency.

### EXAMPLES

Hereinafter, the Examples of the present invention will be described to explain the present invention in more detail. In the examples, both of % and part are weight basis. The following Example 1 is an example of the first aspect, and Example 3 is an example of the second aspect, respectively.

### Example 1

A raw material mixture was prepared by mixing 30 parts of fully hydrogenated high erucic acid rapeseed oil (95% by weight of saturated fatty acid with C18 to C24, 99% by weight of saturated fatty acid with C16 to C24, 56% by weight of saturated fatty acid with C20 to 24, and 82.7% of saturated fatty acids with C20 to 24 at the 1, 3-positions in the constituent fatty acid) as the (a) raw material fat and 70 parts of oleic acid ethyl ester (81% by weight of oleic acid ethyl ester content) as the (b) raw material fatty acid. The mixture was bleached and dehydrated by a known method, and then subjected to the interesterification reaction using a 1,3-position-specific lipase. The interesterification reaction was performed by a batch reaction with 90 ppm of water content in the raw material mixture, 24 hours of reaction time, 53° C. of reaction temperature, and 1% of immobilized lipase relative to the raw material mixture.

After the reaction, the obtained reaction product was separated into a triglyceride fraction and a fatty acid ethyl ester fraction by the distillation. The distillation conditions were: 245 to 250° C. of temperature, and 0.5 to 1.0 torr of degree of vacuum. USU content of the obtained triglyceride fraction was 45% by weight. The triglyceride fraction was further subjected to the solvent fractionation using N-hexane to obtain a fat composition (OStO fat) containing 87% by weight of USU, 58% by weight of OStO, and 0% by weight of OBO as a low-melting point fraction with 45% by weight of yield. In addition, a high-melting point fraction was obtained with 55% by weight of yield as a by-product. This high-melting point fraction contained 19% by weight of SSS and 79% by weight of SSO, and was circulated for reuse as a part of the raw material mixture. The fatty acid ethyl ester fraction obtained by the distillation contained 68% by weight of ethyl oleate. Then, the fatty acid ethyl ester fraction was separated into the low-boiling point fraction and the high-boiling point fraction at the subsequent rectification step. Conditions for the rectification were 238 to 241° C. of temperature and 1.1 to 1.3 torr of the degree of vacuum. The obtained low-boiling point fraction contained 83% by weight of oleic acid ethyl ester. The low-boiling point fraction had a quality almost equivalent to the (b) raw material fatty acid containing 81% by weight of ethyl oleate and was substituted as a part of the (b) raw material fatty acid ethyl ester for reuse to the next interesterification reaction. That is, OStO fat was efficiently produced by circulating the unreacted ethyl oleate for reuse. In addition, the obtained high-boiling point fraction contained 83% by weight of behenic acid ethyl ester.

### Example 2

A raw material mixture was prepared by mixing 30 parts of interesterified oil produced by reacting a fully hydrogenated high erucic acid rapeseed oil using a sodium methylate as a catalyst by an ordinary method (99% by weight of S and 56% by weight of saturated fatty acid with C20 to 24 in the constituent fatty acid) as the (a) raw material fat and 70 parts of oleic acid ethyl ester fraction (81% by weight of oleic acid ethyl ester) as the (b) raw material fatty acid. The mixture was bleached and dehydrated by a known method, and then subjected to interesterification reaction using 1,3-position-specific lipases under the same conditions as Example 1. After the reaction, the obtained reaction product was separated into a triglyceride fraction and a fatty acid ethyl ester fraction by the distillation. The distillation conditions were same as Example 1. USU content of the obtained triglyceride fraction was 42% by weight. The triglyceride fraction was further subjected to the solvent fractionation using N-hexane to obtain a fat composition (OStO/OBO fat) containing 87% by weight of USU, 29% by weight of OStO, and 37% by weight of OBO as a low-melting point fraction with 45% by weight of yield. In addition, a high-melting point fraction was obtained with 55% by weight of yield as a by-product. This high-melting point fraction contained 13% by weight of SSS and 86% by weight of SSO, and was circulated for reuse as a part of the raw material mixture. The fatty acid ethyl ester fraction obtained by the distillation contained 66% by weight of ethyl oleate. Then, the fatty acid ethyl ester fraction was separated into the low-boiling point fraction and the high-boiling point fraction at the subsequent rectification step. Conditions for the rectification were same as Example 1. The obtained low-boiling point fraction contained 83% by weight of oleic acid ethyl ester. The low-boiling point fraction had a quality almost equivalent to the (b) raw material fatty acid containing 81% by weight of ethyl oleate and was substituted as a part of the (b) raw material fatty acid ethyl ester for reuse to the next interesterification reaction. That is, OStO/OBO fat was efficiently produced by circulating the unreacted ethyl oleate for reuse. In addition, the obtained high-boiling point fraction contained 75% by weight of behenic acid ethyl ester.

### Comparative Example 1

A raw material mixture was prepared by mixing 30 parts of fully hydrogenated rapeseed oil (99% by weight of saturated fatty acid with C16 to C24, 2.2% by weight of saturated fatty acid with C20 to 24, and 4% by weight of palmitic acid in the constituent fatty acid) as the (a) raw material fat and 70 parts of oleic acid ethyl ester (81% by weight of oleic acid ethyl ester). The mixture was bleached and dehydrated by a known method, and then subjected to interesterification reaction using 1,3-position-specific lipases under the same conditions as Example 1. After the reaction, the obtained reaction product was separated into a triglyceride fraction and a fatty acid ethyl ester fraction by the distillation. The distillation conditions were same as Example 1.

USU content of the obtained triglyceride fraction was 43% by weight. The triglyceride fraction was further subjected to the solvent fractionation using N-hexane to obtain a fat composition (OStO fat) containing 87% by weight of USU, 58% by weight of OStO, and 0% by weight of OBO as a low-melting point fraction with 48% by weight of yield. The fatty acid ethyl ester fraction obtained by the distillation contained 65% by weight of ethyl oleate, but did not substantially contain saturated fatty acid with C20 to 24. Accordingly, the fatty acid ethyl ester fraction failed to be separated into a low-boiling point fraction and a high-boiling point fraction at the subsequent rectification process, remaining 65% by weight of oleic acid ethyl ester. That is, due to the difference from the (b) raw material fatty acid in quality, the fatty acid ethyl ester fraction was failed to be substituted as the (b) raw material fatty acid for reuse at the subsequent interesterification reaction. Thus, OStO fat was not efficiently produced.

### Example 3

A raw material mixture was prepared by mixing 30 parts of fully hydrogenated palm mid fraction (99% by weight of saturated fatty acid with C16 to C24, 57% by weight of palmitic acid, and 82.7% of palmitic acid content at the 1, 3-positions in the constituent fatty acid) as the (a) raw material fat and 70 parts of oleic acid ethyl ester (81% by weight of oleic acid ethyl ester) as (b) the raw material fatty acid. The mixture was bleached and dehydrated by a known method, and then subjected to the interesterification reaction using a 1,3-position-specific lipase. The interesterification reaction was performed by a batch reaction with 90 ppm of water content in the raw material mixture, 24 hours of reaction time, 53° C. of reaction temperature, and 1% of immobilized lipase relative to the raw material mixture.

After the reaction, the obtained reaction product was separated into a triglyceride fraction and a fatty acid ethyl ester fraction by the distillation. The distillation conditions were: 235 to 240° C. of temperature, and 0.5 to 1.0 torr of degree of vacuum. USU content of the obtained triglyceride fraction was 43% by weight. The triglyceride fraction was further subjected to the solvent fractionation using N-hexane to obtain a fat composition (OStO fat) containing 87% by weight of USU, and 67% by weight of OStO as a low-melting point fraction. In addition, a high-melting point fraction was obtained as a by-product. This high-melting point fraction was circulated for reuse as a part of the raw material mixture. The fatty acid ethyl ester fraction obtained by the distillation was separated into the low-boiling point fraction and the high-boiling point fraction at the subsequent rectification step. Conditions for the rectification were 218 to 221° C. of temperature and 1.1 to 1.3 torr of the degree of vacuum. The obtained high-boiling point fraction contained 83% by weight of oleic acid ethyl ester. The high-boiling point fraction had a quality almost equivalent to the (b) raw material fatty acid containing 81% by weight of ethyl oleate and was substituted as a part of the (b) raw material fatty acid ethyl ester for reuse to the next interesterification reaction. That is, OStO fat was efficiently produced by circulating the unreacted ethyl oleate for reuse. In addition, the obtained low-boiling point fraction contained 88% by weight of palmitic acid ethyl ester.

1. A process for producing a fat composition containing USU, comprising:
(1) a step of mixing (a) raw material fat containing 80% by weight or more of (S) saturated fatty acid with C16 to C24, wherein the (a) raw material fat contains 10 to 70% by weight of saturated fatty acid with C20 to C24 or 10 to 70% by weight of palmitic acid, and (b) raw material fatty acid or lower alcohol ester thereof containing (U) unsaturated fatty acid with C18 as major component;
(2) a step of subjecting the mixture obtained in step (1) to an interesterification reaction using a 1,3-position-specific lipase;
(3) a step of separating the fat composition containing 30% by weight or more of USU as a triglyceride fraction, and a fraction of fatty acid or lower alcohol ester thereof; and
(4) a step of separating the unreacted (b) raw material fatty acid or lower alcohol ester thereof, to circulate a part or all of the recovered unreacted (b) raw material fatty acid or lower alcohol ester thereof as the (b) in step (1) for reuse,
wherein USU is a triglyceride whose fatty acids at 1, 3-positions are U and fatty acid at 2-position is S.

2. The process for producing the fat composition according to claim 1, wherein at least one of the separations at step (3) and step (4) is performed by a distillation.

3. The process for producing the fat composition according to claim 1, wherein the (a) raw material fat contains 80% by weight or more of S and 10 to 70% by weight of the saturated fatty acid with C20 to 24 in the constituent fatty acid.

4. The process for producing the fat composition according to claim 3, wherein a part or all of the (a) raw material fat in step (1) is a fully hydrogenated oil of high erucic rapeseed oil, and wherein the (b) raw material fatty acid or lower alcohol ester thereof contains 70% by weight or more of oleic acid.

5. The process for producing the fat composition according to claim 1, wherein the (a) raw material fat contains 80% by weight or more of S and 10 to 70% by weight of palmitic acid in the constituent fatty acid.

6. The process for producing the fat composition according to claim 5, wherein a part or all of the (a) raw material fat in step (1) is a fully hydrogenated oil of palm mid fraction, and wherein the (b) raw material fatty acid or lower alcohol ester thereof contains 70% by weight or more of oleic acid.

7. The process for producing the fat composition according to claim 1, wherein the triglyceride fraction separated in step (3) is fractionated, and a part or all of the obtained high-melting point fraction is circulated for reuse as the (a) in step (1).

8. The process for producing the fat composition according to claim 1, wherein the triglyceride fraction separated in step (3) is fractionated to obtain the fat composition containing 40% by weight or more of USU as a low-melting point fraction or a mid-melting point fraction.
